(19) United States
(12) Patent Application Publication
(10) Pub. No.: US 2024/0050342 A1
(43) Pub. Date: Feb. 15, 2024

(54) PROLIPID COMPOSITION FOR PERSONALIZED BENEFITS AND METHOD FOR USING THE SAME

(71) Applicant: Conopco, Inc., d/b/a UNILEVER, Englewood Cliffs, NJ (US)

(72) Inventors: John Steven BAJOR, Cheshire, CT (US); Brian John DOBKOWSKI, Milford, CT (US); Galina YAROVA, Monroe, CT (US); William F. LATHROP, Plantsville, CT (US); Jason Peter HARCUP, Chester (GB)

(73) Assignee: Conopco, Inc., d/b/a UNILEVER, Englewood Cliffs, NJ (US)

(21) Appl. No.: 18/269,160

(22) PCT Filed: Dec. 21, 2021
(86) PCT No.: PCT/EP2021/086961

§ 371 Date: Jun. 22, 2023

Related U.S. Application Data
(60) Provisional application No. 63/130,444, filed on Dec. 24, 2020.

(30) Foreign Application Priority Data
Feb. 19, 2021 (EP) 21158293.7

(51) Int. Cl. A61K 8/365; A61K 8/67; A61K 8/41; A61K 8/36; A61K 8/34; A61Q 19/08; A61K 8/42

(52) U.S. Cl. CPC A61K 8/365; A61K 8/675; A61K 8/416; A61K 8/361; A61K 8/345; A61Q 19/08; A61K 8/42

(57) ABSTRACT

The present invention is directed to a prolipid composition that penetrates the stratum corneum. More particularly, the invention is directed to a prolipid composition that provides personalized benefits by rapidly restoring the skins natural lipid balance. Such a composition results in elongated fatty acid and/or lipid production deep in the stratum corneum and near the surface of the stratum lucidum. The composition at least comprises a fatty acid, fatty ester and/or an acylglycerol and an activator of a peroxisome proliferator-activated receptor.

### FIELD OF INVENTION

The present invention is directed to a prolipid composition that penetrates the stratum corneum. More particularly, the invention is directed to a prolipid composition that provides personalized benefits by rapidly restoring the skin's natural lipid balance. Such a composition results in elongated fatty acid and/or lipid production deep in the stratum corneum and near the surface of the stratum lucidum. The composition at least comprises a fatty acid, fatty ester and/or an acylglycerol and an activator of a peroxisome proliferator-activated receptor. Surprisingly, and in addition to rapid personalized fatty acid and lipid balance restoration, the composition results in a long-lasting skin resilience barrier that is suitable to improve skin characteristics, deliver skin benefit agents and protect the skin from environmental contaminants while simultaneously sealing the skin for long lasting moisturization.

### BACKGROUND TO THE INVENTION

Human skin is an important barrier between the body and the environment. Critical for the barrier function of the skin is the lipid matrix found in the stratum corneum. A healthy matrix within the stratum corneum is one which is rich in about an equimolar mixture of ceramides (45-50% by weight), cholesterol (25% by weight) and fatty acids (10-15% by weight) whereby other lipids, like cholesterol sulfate, make up the balance, Kathi C. Madison, Barrier Function of the Skin: “La Raison d'Etre” of the Epidermis, The Society for Investigative Dermatology, Inc. (2003). These fatty acids and waxy lipids regulate the water barrier and retention capacity of skin and they also limit the entry of pollutants and microorganisms that contact skin from the environment. Moreover, the production of ceramides in the skin results in the generation of sphingosines that improve a variety of skin characteristics.

Unfortunately, as consumers age levels of such fatty acids and lipids decline which invariably results in skin that is not supple, dry and even wrinkled. Depletion of the skin's lipid matrix also causes the skin to become more sensitive to contaminants in the environment and often is associated with conditions such as eczema, atopic dermatitis and psoriasis.

Efforts have been made to fortify the fatty acids and lipid matrix of skin. However, penetration of lipids deep into the stratum corneum has been proven to be difficult as these waxy materials are large and not easily transported through the skin's outermost pores. In the absence of penetration, the benefit realized with conventional approaches is short lived, at best.

It is of increasing interest to develop a topical care (i.e., prolipid) composition that replenishes the skin's fatty acids and natural lipid matrix deep within the stratum corneum in order to produce a long lasting skin resilience barrier that is suitable to improve skin characteristics, deliver skin benefit agents and protect the skin from environmental contaminants while simultaneously sealing the skin for long lasting moisturization. This invention, therefore, is directed to a prolipid composition that penetrates into the stratum corneum. Such a composition unexpectedly results in enhanced fatty acid elongation and/or lipid production deep in the stratum corneum and near the surface of the stratum lucidum wherein the same at least comprises a fatty acid, fatty ester and/or an acylglycerol and an activator of a peroxisome proliferator-activated receptor. The composition surprisingly results in a long-lasting skin resilience barrier that is suitable to improve the overall health and appearance of skin.

Additional Information

Efforts have been disclosed for making topical compositions that deliver benefit agents to the skin. In U.S. Pat. No. 6,423,325, topical compositions comprising petroselinic acid and/or docosahexaenoic acid and an activator of peroxisome proliferator activated receptors sub-type alpha are described.

Other efforts have been disclosed for making topical compositions that deliver benefits to the skin. In U.S. Pat. No. 7,160,560, a composition with components that support the skin's collagen and lipid system is described.

Even other efforts have been disclosed for making topical compositions. In U.S. Pat. No. 9,539,190, compositions with aminosilicones and 12-hydroxystearic acid are described.

Still other efforts have been disclosed for making topical compositions. In U.S. Pat. No. 8,613,939, compositions with 12-hydroxystearic acid and ethoxylated hydrogenated castor oil are described.

None of the additional information describes a composition that is suitable to improve the overall health and appearance of skin as defined by the claimed invention. Particularly, none of the additional information describes the unexpected enhancement of personalized fatty acid elongation and/or lipid production resulting from the presence of a peroxisome proliferator-activated receptor in a prolipid composition.

### SUMMARY OF THE INVENTION

In a first aspect, the present invention is directed to a composition comprising:
1) a hydrocarbon source comprising a C₆ to C₁₈ fatty acid, ester and/or an acylglycerol thereof;
2) at least one activator of a peroxisome proliferator-activated receptor;
3) an elongation source comprising a C₂ to C₁₈ alcohol, saccharide, C₃ to C₈ polyol, amino acid or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
   the composition having a C₂₀ to C₃₆ elongated fatty acid, lipid or both after topical application.

In a second aspect, the present invention is directed to a method for treating skin comprising the steps of:
A) applying to skin in need of treatment a composition comprising:
1) a hydrocarbon source comprising a C₆ to C₁₈ fatty acid, ester and/or an acylglycerol thereof;
2) at least one activator of a peroxisome proliferator-activated receptor;
3) an elongation source comprising a C₂ to C₁₈ alcohol, saccharide, C₃ to C₈ polyol, amino acid or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
B) generating a C₂₀ to C₃₆ elongated fatty acid, lipid or both after applying the composition to the skin, the C₂₀ to C₃₆ elongated fatty acid, lipid or both is/are produced in the stratum corneum of the skin and the lipid is a ceramide, cholesterol and/or a mixture thereof.

In a third aspect, the present invention is directed to a method for the personalized treatment of skin deficient in fatty acid, ceramide and/or cholesterol comprising the steps of:
A) applying to skin in need of fatty acid, ceramide and/or cholesterol a composition comprising:
1) a hydrocarbon source comprising C₆ to C₁₈ fatty acid, ester and/or an acylglycerol thereof;
2) at least one activator of a peroxisome proliferator-activated receptor;
3) an elongation source comprising C₂ to C₁₃ alcohol, saccharide, C₃ to C₈ polyol, amino acid or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
B) generating a C₂₀ to C₃₆ elongated fatty acid, lipid or both after applying the composition to the skin; and
C) restoring C₂₀ to C₃₆ fatty acid, ceramide and/or cholesterol levels in the skin to preferably yield an equimolar mixture of the same wherein the C₂₀ to C₃₆ elongated fatty acid, lipid or both is/are produced in the stratum corneum of the skin.

In a fourth aspect, the present invention is directed to a use of the composition of the first aspect of the invention to provide an antiaging benefit to skin by providing a C₂₀ to C₃₆ elongated fatty acid, lipid or both to the stratum corneum.

In a fifth aspect, the present invention is directed to a use of an activator of a peroxisome proliferator-activated receptor in a topical composition to enhance fatty acid elongation, lipid production or both in the stratum corneum.

In a sixth aspect, the present invention is directed to a method for elongating fatty acid and/or making a ceramide by combining a hydrocarbon source, an elongation source and an activator of a peroxisome proliferator-activated receptor to make a composition and combining the composition with an enzyme (the body's fatty acid elongases (ELOVL1-7) in the skin.

All other aspects of the present invention will more readily become apparent from the description and examples which follow.

Skin, as used herein, means skin on the feet, face, neck, chest, arms (including under arms), hands, legs, buttocks, back and scalp (including hair). The composition of the present invention (i.e., end use composition ready to apply and leave-on, or wash off) includes creams, lotions, serums, gels, balms, deodorants and antiperspirants (including aerosols), shampoos, mousses, conditioners, bars and liquid wash products. In one embodiment, the end use composition of the present invention is a personal wash composition, a face wash product or a leave-on product, such as a cosmetic cream or lotion to be applied to the face, body or hands. In another embodiment, the end use composition is a cosmetic leave-on product suitable to reduce wrinkles, moisturize and/or result in skin having an even colour or tone. Cosmetic (i.e., skin care) means herein regulating and/or improving cosmetic qualities of the skin. These qualities are subject to regulation and/or improvement both in healthy subjects as well as those which present diseases or disorders of the skin (such as psoriasis, lichen planus, folliculitis or atopic dermatitis). Examples of skin care (cosmetic) benefits in the context of this invention include providing a smoother, more even texture; improving the elasticity or resiliency of the skin; improving the firmness of the skin; improving the hydration status or moisturization of the skin; improving skin barrier properties; and reducing the appearance of hyperpigmentation, redness or skin blotches.

In an embodiment of the invention and as used herein the C₆ to C₁₈ fatty acid, ester and/or acylglycerol used is a hydrocarbon source that may be unsubstituted or substituted (i.e., with from 1 to 3 functional groups which are —OH and/or —NH₂ on the hydrocarbon), saturated, unsaturated (typically no more than 2 double bonds), linear or branched; however, the hydrocarbon source is preferably linear, unsubstituted and saturated. In another embodiment of the invention, such a hydrocarbon source is not substituted with a heteroatom, and therefore, does not comprise hydroxy groups. Elongated fatty acid means a fatty acid having at least 2 more carbons in its chain from the initial hydrocarbon source in the composition and preferably

at least a twenty-carbon chain. Hydrocarbon source means the fatty chain provided (i.e., by the C₆ to C₁₈ fatty acid, ester and/or acylglycerol) in the composition of this invention that is suitable to extend in the stratum corneum after being applied topically to skin. Elongation source means the alcohol, saccharide, polyol and/or amino acid suitable to provide carbon to the hydrocarbon source during elongation. The composition (i.e., end use composition) of the present invention is a prolipid composition, meaning a composition that is suitable to restore the fatty acid content, ceramide and/or cholesterol levels of consumers in need of one or all of the same to an equimolar mixture where equimolar mixture means within 20%, and preferably, within 15%, and most preferably, within 10% of an equimolar mixture. Therefore, the prolipid composition of the present invention is one which results in personalized results in that it can restore fatty acid content, ceramide and/or cholesterol levels, depending on what the consumer's matrix content needs. Acylglycerol, as used herein, means an ester formed from glycerol and fatty acid (i.e., a mono-, di, or tri-glyceride). A C₆ to C₁₈ fatty acid, ester and/or an acylglycerol thereof (i.e., derivative of fatty acid) means a C₆ to C₁₈ fatty chain is the chain in the acid, ester and/or acylglycerol used in the invention. Hydrocarbon source, elongation source and an activator of a peroxisome proliferator-activated receptor are ingredients in the invention and included in the term components of the invention.

Rapidly restoring the skin's natural lipid balance means enhancing the formation of elongated fatty acid and/or ceramide and not impeding cholesterol formation within the stratum corneum by including at least one activator of a peroxisome proliferator-activated receptor in the composition of this invention. Long-lasting skin resilience barrier means providing a fatty acid and lipid balance for at least 1.5 weeks, and preferably, for at 2.5 weeks, and most preferably, for at least 4 weeks after topically applying the composition.

Unless explicitly stated otherwise, all ranges described herein are meant to include all ranges subsumed therein. The term comprises is meant to encompass the terms consisting essentially of and consisting of. For the avoidance of doubt, a composition comprising fatty acid, an activator of a peroxisome proliferator-activated receptor and polyol, is meant to include a composition consisting essentially of and consisting of such components. As to the percentages used herein, the same are meant to be by weight unless noted otherwise. Except in the operating and comparative examples, or where otherwise explicitly indicated, all numbers used in this description indicating amounts, or ratios of materials and/or use thereof are to be understood as modified by the word “about”.

### DETAILED DESCRIPTION OF THE INVENTION

The only limitation with respect to the C₆ to C₁₈ fatty acid, ester and/or acylglycerol thereof (i.e., hydrocarbon source) that may be used in the present invention is that the same results in a composition that is homogeneous and free of precipitate. Illustrative examples of the fatty acids suitable for use include hexanoic, heptanoic, octanoic, nonanoic, lauric, myristic, palmitic, stearic, behenic, oleic, linoleic, linolenic, isostearic acid or mixtures thereof. Derivatives of the same may be used and these include any ester and/or acylglycerol thereof and resulting from, for example, a dehydration reaction like a Fischer esterification in the presence of alcohols. Preferred acylglycerols are those having hydrocarbon chains of lauric, myristic, palmitic and/or stearic acid. The ester derivative, when used, will typically result from an esterification that uses a C₁-C₆ alcohol as a reagent. Fatty esters often desirable for use include palmitic acid methyl ester, palmitic acid ethyl ester, palmitic acid hexyl ester, isopropyl palmitate, isopropyl isostearate, isopropyl myristate or a mixture thereof.

As to the acylglycerol, the hydrocarbon chains may be the same or different and they are typically C₆ to C₁₈ chains. An often preferred acylglycerol used in this invention is a triglyceride esterified with palmitic acid.

Typically, C₆ to C₁₈ fatty acid, ester and/or acylglycerol thereof will make up from 0.05 to 8.0%, and preferably, from 1 to 6%, and most preferably, from 1.5 to 4.5% by weight of the composition. In an embodiment of the invention, the composition will have from 1.5 to 4%, and still another embodiment, from 1.5 to 3.5% by weight C₆ to C₁₈ fatty acid, ester and/or acylglycerol thereof. In yet another embodiment of the invention, the hydrocarbon source is at least 50%, and preferably, at least 75 to 99% by weight C₆ to C₁₈ fatty acid based on total weight of hydrocarbon source in the composition. In even another embodiment, the hydrocarbon source is all (100% by weight) C₆ to C₁₈ fatty acid based on total weight of hydrocarbon source in the composition.

Regarding the activator of a peroxisome proliferator-activated receptor (PPAR activator) that is suitable for use, the same is typically 12-hydroxystearic acid, cis parinaric acid, trans-7-octadecenoic acid, cis 5,8,11,14,17 eicosapentanoic acid, cis-4,7,10,13,16,19 docosahexenoic acid, conjugated linoleic acid (c9,t11), columbinic acid, linolenelaidic acid, ricinolaidic acid, stearidonic acid, 2-hydroxystearic acid, alpha-linolenic acid, arachidonic acid, cis-11,14-eicosadienoic acid, conjugated linoleic (t10,c12), conjugated linoleic acid (t9,t11), conjugated linoleic acid (50:50 mix of c9, t11 and t10 c12), coriander acids, linolelaidic acid, monopetroselinic acid, petroselinic acid, ricinoleic acid, stearolic acid, thuja extract or trans vaccenic acid.

Even other PPAR activators suitable for use include cis-11,14,17 eicosatrienoic acid, cis-5 eicosenoic acid, cis-8,11,14 eicosatrienoic acid, hexadecatrienoic acid, palmitoleic acid, petroselaidic acid, trans trans farnesol, cis 13, 16 docosadienoic acid, cis vaccenic acid, cis-11 eicosenoic acid, cis-13,16,19 docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, petroselinyl alcohol, phytanic acid, pinolenic acid, trans-13-octadecenoic acid or tridecyl salicylic acid (TDS).

Still other PPAR activators suitable for use include biochanin A (red clover phytoestrogen), chromolaena odorata extract, pomegranate saponifiable hydrolysable extract, buglossoides (stearidonic plant extract) or zanthalene (extract from Sichuan peppercorn), whereby it is within the scope of the invention to use a mixture of any of the herein noted PPAR activators. In an embodiment of the invention, the PPAR activator is petroselinic acid, conjugated linoleic acid, 12-hydroxystearic acid, ricinoleic acid or a mixture thereof. In yet another embodiment of the invention, the PPAR activator is 12-hydroxystearic acid.

Typically, PPAR activator will make up from 0.05 to 8.0% and preferably, from 1 to 6%, and most preferably, from 1.5 to 4.5% by weight of the composition. In an embodiment of the invention, the composition will have from 1.5 to 4%, and in still another embodiment, from 1.5 to 3.5% by weight PPAR activator. In even another embodiment of the invention, the C₆ to C₁₈ fatty acid used is palmitic acid and the PPAR activator is 12-hydroxystearic acid.

Illustrative examples of the C₂ to C₁₈ alcohols suitable for use in the present invention include ethyl alcohol, lauryl alcohol, cetyl alcohol, stearyl alcohol or a mixture thereof. As to the saccharides suitable for use, these include those that are soluble in the composition of the present invention. Illustrative examples of the saccharides suitable for use include glucose (dextrose monohydrate), galactose, sucrose, lactose, fructose, mixtures thereof or the like.

Regarding the polyols suitable for use, illustrative examples include sorbitol, glycerol, mannitol, xylitol, maltitol or mixtures thereof. Other polyols suitable for use include propylene glycol, dipropylene glycol, polypropylene glycol, polyethylene glycol, hydroxypropyl sorbitol, hexylene glycol, 1,3-butylene glycol, isoprene glycol, 1,2,6-hexanetriol, ethoxylated glycerol, propoxylated glycerol or mixtures thereof.

In an embodiment of the invention, the polyol used is at least 50% by weight glycerol, based on total weight of the polyol used in the composition. In another embodiment of the invention, the polyol used is all glycerol (100% by weight).

Amino acids suitable for use in the compositions of this invention include serine, threonine, cysteine, asparagine, glutamine, tyrosine or mixtures thereof.

The amount of C₂ to C₁₈ alcohol, saccharide and/or amino acid will typically make up from 0.01 to 7%, and preferably, from 0.5 to 6%, and most preferably, from 1 to 4.5% by weight of the composition. Polyol when used in the composition of the present invention (i.e., as the elongation source) typically makes up from about 0.01 to 35%, and preferably, from 1 to 25%, and most preferably, from 1.5 to 15% by weight, based on total weight of the of the composition. In an embodiment of the invention, the elongation source is at least 50% by weight polyol based on total weight of the elongation source in the composition. In another embodiment, the elongation source used is at least 50 to 99% by weight glycerol based on total weight of elongation source in the composition. In still another embodiment, the elongation source is 100% by weight glycerol. In even another embodiment, the composition comprises from 2 to 12%, and preferably, from 2 to 8% elongation source. In yet another embodiment, when the elongation source is a C₂ to C₁₈ alcohol, the composition comprises from 0.1 to 5% by weight of alcohol. In but another embodiment, when the elongation source is an amino acid, the composition comprises from 0.05 to 5%, and preferably, from 0.05 to 3% by weight of amino acid.

In an especially preferred embodiment, the prolipid composition of the present invention has a hydrocarbon source (HS) which is palmitic acid, an elongation source (ES) which is glycerol and a PPAR activator which is 12-hydroxystearic acid. In another especially preferred embodiment, the weight ratio in the prolipid composition of HS:ES:PPAR activator is 15:70 to 15:70 to 15:70, and preferably, 20:60 to 20:60 to 20:60, and most preferably, from 25:50 to 25:50 to 25:50, including all weight ratios subsumed therein.

It has been unexpectedly found that fatty acid elongation and ceramide production are enhanced in the stratum corneum when hydrocarbon and elongation sources are provided with a PPAR activator as described in this invention. Not wishing to be bound by theory, the body's fatty acid elongases (ELOVL1-7) would, as normally expected, catalyze the rate limiting step of the fatty acid elongation cycle. The elongated fatty acid produced with the composition of the invention is typically a C₂₀ to C₃₆ fatty acid, and preferably, very long-chain fatty acid (VLCFA) such as a C₂₂ to C₃₂ fatty acid, and most preferably, a C₂₄ to C₃₀ fatty acid. It is expected that ceramide

(sphingosine and fatty acid) production would likely include condensation of fatty acid with amino acid, like serine, to form 3-keto dihydrosphingosine. The same is reduced with a cellular 3-ketosphinganine reductase to yield dihydrosphingosine, and further acylated to dihydroceramide through the activity of ceramide synthase enzymes. Unsaturation in the original fatty acid portion of this molecule via the enzyme dihydroceramide desaturase completes production of ceramide whereby such synthesis is unexpectedly enhanced with topical application of the present prolipid composition of the present that is formulated with a PPAR activator.

The skin's cholesterol synthesis is known to be associated with an increase in activity, protein and mRNA levels of key enzymes in the body's cholesterol synthetic pathway and is unimpeded with the composition of this invention.

There is generally no limitation with respect to the type of end use composition (i.e., the prolipid composition) that may comprise the components of the present invention.

Prolipid compositions that are suitable to deliver to skin hydrocarbon and elongation sources with a PPAR activator will typically include cosmetically acceptable carrier components. Water is the most preferred carrier. Amounts of water may range from 1 to 95%, and preferably from 5 to 85%, and most preferably, from 35 to 80%, and optimally, from 40 to 75% by weight, based on total weight of the prolipid composition. Ordinarily the prolipid compositions of this invention will be water and oil emulsions, most preferably, of the oil-in-water variety. Water-in-oil emulsions, and especially, those generally classified as water-in-oil and high internal phase emulsions are, however, an option. Illustrative examples of the high internal phase emulsions suitable to include the components of this invention are described in commonly owned U.S. Patent Application Publication No. 2008/0311058 and U.S. Pat. No. 8,425,882, the disclosures of which are incorporated herein by reference.

Other cosmetically acceptable carriers suitable for use in this invention may include mineral oils, silicone oils, synthetic or natural esters, and alcohols. Amounts of these materials may range from 0.1 to 50%, and preferably, from 0.1 to 30%, and most preferably, from 1 to 20% by weight of the composition, including all ranges subsumed therein.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, and preferably, from 4 to 5 silicon atoms. Linear volatile silicone materials generally have viscosities of less than 5 centistokes at 25° C. while cyclic materials typically have viscosities of less than 10 centistokes (measured with a Brookfield Viscometer, RV No. 3 spindle at 20 PRM, standardized to mineral oil and at 25° C.).

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone, including crosspolymers and elastomers) with viscosities of from 5 to 100,000 centistokes at 25° C.

An often-preferred silicone source is a cyclopentasiloxane and dimethiconol solution.

Among suitable esters are:
(1) Esters of fatty acids like isononyl isonanonoate, oleyl myristate, oleyl stearate, and oleyl oleate;
(2) Ether-esters such as fatty acid esters of ethoxylated fatty alcohols;
(3) Polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid esters, sorbitan fatty acid esters, and polyoxyethylene sorbitan fatty acid esters;
(4) Wax esters such as beeswax, spermaceti, myristyl myristate, stearyl stearate; and
(5) Sterol esters, of which soya sterol and cholesterol fatty acid esters are examples thereof.

Emulsifiers (or surfactants) may be present in the prolipid compositions comprising the components of the present invention. Total concentration of the emulsifier, when used, may range from 0.1 to 30%, and preferably, from 2 to 20%, and most preferably, from 1 to 9% by weight of the composition. The emulsifier may be selected from the group consisting of anionic, nonionic, cationic and amphoteric components. Particularly preferred nonionic components are those with a C₁₀ to C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, cetyl alcohol as well as emulsifying/thickening additives like hydroxyethylacrylate/sodium acryloyldimethyl taurates copolymer/squalene and mixtures thereof.

Preservatives can be incorporated into the prolipid compositions comprising the components of this invention to assist against the growth of potentially harmful microorganisms. Suitable traditional preservatives for use in the compositions of this invention include alkyl esters of para-hydroxybenzoic acid. Other preservatives include hydantoin derivatives, propionate salts, and a variety of quaternary ammonium compounds. Often preferred preservatives are iodopropynyl butyl carbamate, phenoxyethanol, methyl paraben, propyl paraben, imidazolidinyl urea, sodium dehydroacetate, sodium benzoate and benzyl alcohol. Especially preferred additives suitable to be employed with preservatives used in this invention are 1,2-alkanediols (like 1,2-octanediol and 1,2 hexanediol) as well as commonly used fragrance oils (added separately or with the fragrance in the prolioid composition) like eugenol, coumarin, linalyl acetate, citronellal, iris concentrate, terpinyl acetate, pinenes (alpha and beta pinene) and citronellol.

Typically, the preservative, vicinal diol and/or fragrance oil will not make up more than 2%, and preferably, not more than 1.5%, and most preferably, not more than 1.0% by weight of the composition of the present invention. In an embodiment of this invention from 0.15 to 0.95% by weight preservative, vicinal diol and/or fragrance component is collectively used, based on total weight of the composition, including all ranges subsumed therein.

Thickening agents may optionally be included in the prolipid compositions of the present invention. Particularly useful are the polysaccharides. Examples include starches, natural/synthetic gums and cellulosics. Representative of the starches are chemically modified starches such as sodium hydroxypropyl starch phosphate and aluminium starch octenylsuccinate. Tapioca starch is often preferred. Suitable gums include xanthan, sclerotium, pectin, karaya, Arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective

thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel IN100. Polyquaternium 32 and/or 37 are also suitable for use.

Cellulose microfibrils may be used as thickening agents and these include secondary cell wall materials (e.g. wood pulp, cotton), bacterial cellulose, and primary cell wall materials. Often the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

Amounts of the thickener, when used, may range from 0.001 to 5%, and preferably, from 0.1 to 2%, and most preferably, from 0.2 to 0.5% by weight of the composition and including all ranges subsumed therein.

Fragrances, fixatives and abrasives may optionally be used in the compositions that include the components of the present invention. Each of these substances may range from 0.05 to 5%, preferably between 0.1 and 3% by weight of the composition.

In addition to the polyols described herein as elongation sources, quaternary ammonium humectants (moisturizers) may be employed in the prolipid compositions of the present invention. Dihydroxypropyl quaternary ammonium salts of structure AB are often preferred, wherein A is a cationic charged component of the salt AB, and B is an anionic charged component of the salt AB, A has one quaternized nitrogen atom, at least two hydroxyl groups and a molecular weight no higher than about 250 but preferably no higher than about 200, and optimally no higher than 170.

Anionic charged component B may be organic or inorganic with proviso that the material is cosmetically acceptable. Typical inorganic anions are halides, sulfates, phosphates, nitrates and borates. Most preferred are the halides, especially chloride. Organic anionic counter ions include methosulfate, toluoyl sulfate, acetate, citrate, tartrate, lactate (e.g., potassium), gluconate, potassium, sodium and/or magnesium chloride, sea salt, benzenesulfonate or a mixture thereof. The number and charge of negatively charged component B will be sufficient to neutralize the positive charge of component A.

A preferred embodiment of the quaternary ammonium salts is the dihydroxypropyl tri (C₁-C₃ alkyl or hydroxyalkyl) ammonium salts. These salts may be obtained in a variety of synthetic procedures, most particularly by hydrolysis of chlorohydroxypropyltri (C₁-C₃ alkyl or hydroxyalkyl) ammonium salts. Ordinarily the C₁-C₃ alkyl or hydroxyalkyl constituent on the quaternized ammonium group will be methyl, ethyl, n-propyl, isopropyl, hydroxyethyl, hydroxymethyl and mixtures thereof. Particularly preferred is a trimethyl ammonium group known through INCI nomenclature as a “trimonium” group.

A most preferred species is 1,2-dihydroxypropyltrimonium chloride, wherein the C₁-C₃ alkyl is a methyl group. When used, the quaternary ammonium salt makes up from 0.001 to 10%, and preferably, from 0.1 to 6%, and most preferably, from 0.1 to 3% by weight of the prolipid composition.

Other preferred moisturizing agents which may be used in the prolipid compositions of the present invention, especially in conjunction with the aforementioned ammonium salts, include substituted urea like hydroxymethyl urea, hydroxyethyl urea, hydroxypropyl urea; bis(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N,N,N′-tri-hydroxyethyl urea; tetra(hydroxymethyl) urea; tetra(hydroxyethyl) urea; tetra(hydroxypropyl urea; N-methyl, N′-hydroxyethyl urea; N-ethyl-N′-hydroxyethyl urea; N-hydroxypropyl-N′-hydroxyethyl urea and N,N′dimethyl-N-hydroxyethyl urea. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch & Chemical Division of ICI under the trademark Hydrovance. Amounts of substituted urea that may be used in the topical composition of this invention range from 0.01 to 10%, or from 0.5 to 8%, or from 2 to 6% by weight of the composition, when used. Further moisturizing agents for use herein include petrolatum and/or various aquaporin manipulating actives and/or oat kernel flour. Even others include saccharide isomerates like Pentavitin® made commercially available from Royal DSM. These agents, when used, typically make up from 0.01 to 6% by weight of the composition. The prolipid compositions of the present invention may include vitamins. Illustrative vitamins suitable to use include Vitamin A (retinol) as well as retinol esters like retinol palmitate and retinol propionate, Vitamin B₂, Vitamin B₆, Vitamin C, Vitamin D Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of such vitamins when present may range from 0.001 to 10%, and preferably from 0.01% to 3.5%, optimally from 0.1 to 01.5% by weight of the composition.

Other optional additives suitable for use in the compositions of this invention include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol mixtures thereof or the like. Such additives, when used, collectively make up from 0.001 to 12% by weight of the composition, and preferably, from 0.01 to 4% by weight of the composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Salicylic acid is representative of the beta-hydroxycarboxylic acids. Amounts of these materials when present may range from 0.01 to 15% by weight of the composition.

A variety of herbal extracts may optionally be included in compositions of this invention. The extracts may either be water soluble or water-insoluble and carried in a solvent which respectively is hydrophilic or hydrophobic. Water and ethanol are often the preferred extract solvents. Illustrative extracts suitable for use include those removed from green tea, yarrow, chamomile, licorice, aloe vera, grape seed, citrus unshiu, willow bark, sage, rosemary and mixtures thereof. When used, such extracts make up from 0.01 to 5% by weight of the composition.

In an especially preferred embodiment, the prolipid composition of the present invention does optionally comprise at least one of niacin, niacinamide, inositol, hexanicotinate, picolinic acid, picolinamide, 1-methylnicotinamide chloride, N-acetyl-DL-methionine or a mixture thereof. When present, these materials can make up from 0.001 to 5%, and preferably, from 0.01 to 4.5%, and most preferably, from 0.1 to 4% by weight of the composition. In an especially preferred embodiment, the composition of the present invention does comprise niacinamide.

Also optionally suitable for use include materials like chelators (e.g., EDTA), opacifiers (like TiO₂, particle size from 50 to 1200 nm, and preferably, 50 to 350 nm) and exfoliants. Amounts of these materials may range from 0.0001 to 8%, and preferably, from 0.0001 to 2% by weight of the composition.

Sunscreen actives may also be included in the compositions of the present invention. Particularly preferred are materials like ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.1 to 15%, preferably from 0.5 to 10%, optimally from 0.75 to 7% by weight of the composition. Conventional buffers/pH modifiers may be used in the prolipid compositions of the present invention. These include commonly employed additives like sodium hydroxide, potassium hydroxide, hydrochloric acid, citric acid and citrate/citric acid buffers. In an especially preferred embodiment, the pH of the prolipid composition of this invention is from 4 to 10, and preferably, from 4.25 to 7.85, and most preferably, from 5.65 to 7.5. Film forming agents may be used in the compositions of the present invention. While optional, such agents can aid with composition adhering to the surface they are applied to. Film forming agents include those having hydrophilic properties and they include materials comprising polyvinylpyrrolidone (PVP), acrylates, acrylamides, and copolymers thereof. When used, such agents make up from 0.001 to 1% by weight of the composition.

Other optional components suitable for use in the composition are anti-mosquito agents like eucalyptus oil, lavender oil, N,N-diethyl-meta-toluamide (DEET), a mixture thereof or the like. Even other ingredients which may be used include octopirox (piroctone), zinc pyrithione, thymol, terpineol, cetrimonium chloride, benzethonium chloride, benzalkonium chloride, chloroxylenol, triclosan, cetylpyridinium chloride as well as silver compounds including silver oxide, nitrate, sulfate, phosphate, carbonate, acetate, benzoate, a mixture thereof or the like. Even other optional components that may be used in the compositions of the present invention include honokiol, antimicrobial lipids like sapienic acid, palmitoleic acid, sphingosine, dihydrosphingosine, phytosphingosine, and 6-hydroxysphingosine as well as antimicrobial proteins like myeloid antimicrobial proteins. If used, these other components typically make up from 0.001 to 2.6%, and preferably, from 0.01 to 1.2% by weight of the composition.

Another optional additive suitable for use includes hemp oil with 2.5 to 25% by weight cannabigerol and/or cannabidiol at from 0.5 to 10 percent by weight. When used, such oil makes up from 0.0001 to 1.5% by weight of the composition, and preferably, from 0.01 to 1% by weight of the composition.

In an often desired embodiment, the composition of the present invention comprises (from 0.001 to 5% by weight) at least on of retinol palmitate, retinol propionate, coumarin, farnesol, geraniol, climbazole and vanillin. In another embodiment, the composition of the present invention comprises from 0.001 to 4.5%, and preferably, from 0.1 to 3.5% by weight climbazole and retinol palmitate and/or proprionate. When making the prolipid composition of the invention, ingredients/components are mixed with moderate shear, under atmospheric conditions and at a temperature from 20 to 65° C.

Typically, the viscosity of the prolipid composition will be under 25,000 cps. Often the viscosity of the composition will be from 250 to 22,000, and preferably, from 350 to 13,000, and most preferably, from 500 to 10,000 cps. Viscosity may be measured with art recognized instrumentation such as a Brookfield Viscometer RVT, Model D220, using a T-bar spindle D at 5 RPM, 60 seconds at 25° C.

In an embodiment of the invention and when the prolipid composition is a shampoo or wash composition, the same often comprises fatty acid soap or non-soap synthetic surfactant (like isethionates, taurates and/or sulfates). Soap bars suitable for use with the components of the invention can comprise at least 50 to 70% by weight fatty acid soap and up to 25% by weight non-soap synthetic surfactant whereby the bars are typically less than 15% by weight water. Liquid body wash compositions will often comprise soaps and non-soap synthetic surfactants. These often include isethionates, taurates betaines and/or sulfates like sodium lauryl sulfate and sodium lauryl ether sulfate. Prolipid compositions that are deodorants and/or antiperspirant products suitable to include the components of the present invention can be pump sprays, aerosol sprays, roll-ons, sticks, soft solids or gels made with conventional bases. When deodorant products, the same can comprise aluminium, nonaluminum active or both. The composition of the invention is, preferably, a leave on skin composition.

As to packaging, the prolipid composition of the present invention, can be packaged in a spray bottle, or provided as an impregnating wetting agent on cotton swab, wipe, towelette, cosmetic substrate sheet (like those described in U.S. Pat. No. 6,294,182 B1) or the like. As the viscosity is increased with thickening agent, the composition may be provided to consumers in a squeeze bottle as a gel composition or as a customary lotion, cream or wash (i.e., emulsion) from customary stock bottles and jars. Conventional aerosol packaging technologies and including those which utilize air-in-bag discharging canisters, mechanisms and actuators may also be used. It is also within the scope of the invention to include foaming agents (e.g., zwitterionic and/or amphoteric surfactants) so that the composition can be discharged as a foam or mousse.

The prolipid composition of the invention should be supplied with instructions to apply the composition to hair or skin and for the personalized restoration of the stratum corneum's fatty acid content and lipid matrix.

In an embodiment of the invention and when the prolipid composition is a shampoo or wash composition, the same often comprises fatty acid soap or non-soap synthetic surfactant (like isethionates, taurates and/or sulfates). Soap bars suitable for use with the components of the invention can comprise at least 50 to 70% by weight fatty acid soap and up to 25% by weight non-soap synthetic surfactant whereby the bars are typically less than 15% by weight water. Liquid body wash compositions will often comprise soaps and non-soap synthetic surfactants. These often include isethionates, taurates betaines and/or sulfates like sodium lauryl sulfate and sodium lauryl ether sulfate. Prolipid compositions that are deodorants and/or antiperspirant products suitable to include the components of the present invention can be pump sprays, aerosol sprays, roll-ons, sticks, soft solids or gels made with conventional bases. When deodorant products, the same can comprise aluminium, nonaluminum active or both. The composition of the invention is, preferably, a leave on skin composition.

As to packaging, the prolipid composition of the present invention, can be packaged in a spray bottle, or provided as an impregnating wetting agent on cotton swab, wipe, towelette, cosmetic substrate sheet (like those described in U.S. Pat. No. 6,294,182 B1) or the like. As the viscosity is increased with thickening agent, the composition may be provided to consumers in a squeeze bottle as a gel composition or as a customary lotion, cream or wash (i.e., emulsion) from customary stock bottles and jars.

Conventional aerosol packaging technologies and including those which utilize air-in-bag discharging canisters, mechanisms and actuators may also be used. It is also within the scope of the invention to include foaming agents (e.g., zwitterionic and/or amphoteric surfactants) so that the composition can be discharged as a foam or mousse.

The prolipid composition of the invention should be supplied with instructions to apply the composition to hair or skin and for the personalized restoration of the stratum corneum's fatty acid content and lipid matrix.

In an embodiment of the invention and when the prolipid composition is a shampoo or wash composition, the same often comprises fatty acid soap or non-soap synthetic surfactant (like isethionates, taurates and/or sulfates). Soap bars suitable for use with the components of the invention can comprise at least 50 to 70% by weight fatty acid soap and up to 25% by weight non-soap synthetic surfactant whereby the bars are typically less than 15% by weight water. Liquid body wash compositions will often comprise soaps and non-soap synthetic surfactants. These often include isethionates, taurates betaines and/or sulfates like sodium lauryl sulfate and sodium lauryl ether sulfate. Prolipid compositions that are deodorants and/or antiperspirant products suitable to include the components of the present invention can be pump sprays, aerosol sprays, roll-ons, sticks, soft solids or gels made with conventional bases. When deodorant products, the same can comprise aluminium, nonaluminum active or both.

The following Examples are provided to further illustrate an understanding of the invention. The Examples are not intended to limit the scope of the claims.

### Example 1

In this experiment, a standard topical base composition was charged with 3% by weight deuterated d31palmitic acid and a second base was charged with 3% by weight deuterated d31palmitic acid in combination with 3% by weight 12-hydroxystearic acid. Both compositions were applied to separate human skin discards. Biopsies were taken, placed on collagen and incubated at 37° C. with about 5% carbon dioxide. Samples were harvested after 5 days, extracted, and fractionated to obtain ceramide fractions. Elongation of deuterated d31Palmitic acid (dC16:0) with and without the PPAR activator 12-hydroxystearic acid was investigated in an ex vivo human skin model. The ceramide fraction obtained was hydrolyzed to release deuterated sphingosine and analyzed via liquid chromatography/tandem mass-spectrometry (LC/MS/MS). This technique was used to identify and measure “precursor-to-product” ion transitions specific to deuterated sphingosine, and therefore, to track sphingosine formation from deuterated palmitic acid applied to skin discards. Unexpectedly, the amount of resulting deuterated sphingosine increased in the presence the PPAR activator. The numbers in units (“u”) provided depict the signal abundance of the biomolecules looked and accounted for in a sample as determined by LC/MS/MS. In Example 1, the biomolecule is sphingosine and its abundance reflects production of ceramides from deuterated palmitic acid in the presence and absence of PPAR activator. In Example 2 and Example 3, the biomolecules are elongated fatty acids.

Deuterated Sphingosine

No PPAR with deuterated palmitic acid 36,958 u

PPAR with deuterated palmitic acid 57,810 u

### Example 2

In this experiment, EpiDerm in vitro 3D skin equivalents of keratinocytes (commercially available from Matek) were used as a model system to investigate bioconversions in human skin. The skin equivalents were topically treated with deuterated d23-Lauric acid (dC12:0) in propylene glycol:ethanol (70:30), one time per day for seven days with concentrations in the range of 400 ng/cm² to 5000 ng/cm². Samples were harvested, extracted and analyzed via liquid chromatography/tandem mass-spectrometry (LC/MS/MS) for deuterated d23-Lauric acid elongation. Precursor-to-product (i.e., elongation) ion transitions, specific to deuterated elongated fatty acids derived from d23 Lauric acid, were identified and measured. The first column depicts the signal for lauric acid provided to the EpiDerm prior to elongation.

Confirmed was the ability of dC12:0 to convert to dC16:0 via fatty acid synthase enzyme, followed by further elongation of dC16:0 to deuterated Lignoceric acid C24:0 via elongases where the enzymes were present in the skin equivalents.

Elongation of deuterated Lauric acid (dC12:0)

| Sample name | d23 C12:0 | d23 C14:0 | d23 C16:0 | d23 C18:0 | d23 C20 | d23 C22:0 | d23 C24:0 |
|---|---|---|---|---|---|---|---|
| 5000 ng/cm2 dC12:0 | 528578 | 53655 | 1026969 | 1355969 | 227595 | 513274 | 758093 |
| 1000 ng/cm2 d12:0 | 51904 | 7481 | 338885 | 467624 | 54050 | 128640 | 187974 |
| 400 ng/cm2 d12:0 | 24559 | 4670 | 46204 | 140851 | 20848 | 45885 | 68863 |

### Example 3

In this experiment the elongation of deuterated d31-Palmitic acid (dC16:0) with and without PPAR activator 12-hydroxystearic was investigated in a neonatal keratinocyte model.

Keratinocytes were grown in an EpiLife™ medium (with calcium, from Thermo Fisher Scientific) in six well plate formats until confluency. Confluent keratinocytes were differentiated with CaCl2 treatment.

The cells in wells deplete of PPAR activator were treated with the substrate: 1 uM of d31-Palmitic acid (dC16:0) every second or third day until harvested on day 7. The remainder of the samples were treated with 1 uM of the aforementioned dC16:0 and 12 hydroxystearic acid at the concentrations shown in the tables.

Samples were extracted and analyzed via liquid chromatography/tandem mass-spectrometry (LC/MS/MS) for deuterated d31-Palmitic acid (dC16:0) elongation. Precursor-to-product ion transitions, specific to deuterated elongated fatty acids derived from d31-Palmitic acid, were identified and measured.

Unexpectedly, it was demonstrated that an activator of a peroxisome proliferator-activated receptor like 12-hydroxystearic acid (12-HSA) boosts and enhances the formation of elongated fatty acids from d31C16:0 to d31C26:0 when compared to samples deplete of the activator.

The data provided, therefore, surprisingly shows that compositions made according to this invention will result in enhanced formation of elongated fatty acid, lipid or both after topical application. The numbers provided in the table below are in units “u” as defined above.

Elongation of deuterated Palmitic acid
(dC16:0) with and without 12 HSA

| | No 12 | 12HSA | 12HSA | 12HSA | 12HSA |
|---|---|---|---|---|---|
| | HSA | 100 uM | 30 uM | 10 uM | 3 uM |
| dC16:0 | 646987 | 677241 | 921997 | 503533 | 486506 |
| dC18:0 | 663577 | 750585 | 792317 | 713499 | 710249 |
| dC20:0 | 11233 | 75060 | 18245 | 13666 | 11478 |
| dC22:0 | 90496 | 201806 | 131062 | 98634 | 94091 |
| dC24:0 | 278426 | 774622 | 470314 | 306176 | 294398 |
| dC26:0 | 60009 | 219006 | 109818 | 62141 | 60180 |

1. A composition comprising:
1) a hydrocarbon source comprising a C₆ to C₁₈ fatty acid, ester and/or acylglycerol thereof;
2) at least one activator of a peroxisome proliferator-activated receptor;
3) an elongation source comprising a C₂ to C₁₈ alcohol, saccharide, C₃ to C₈ polyol or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
the composition having C₂₀ to C₃₆ elongated fatty acid, lipid or both after topical application.

2. A method for treating skin comprising the steps of:
A) applying to skin in need of treatment a composition comprising:
1) a hydrocarbon source comprising a C₆ to C₁₈ fatty acid, ester and/or acylglycerol thereof;
2) at least one activator of a peroxisome proliferator-activated receptor;
3) an elongation source comprising a C₂ to C₁₈ alcohol, saccharide, C₃ to C₈ polyol or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
B) generating a C₂₀ to C₃₆ elongated fatty acid, lipid or both after applying the composition to the skin,
the C₂₀ to C₃₆ elongated fatty acid, lipid or both is/are produced in the stratum corneum of the skin and the lipid is cholesterol, ceramide and/or a mixture thereof.

3. A method for the personalized treatment of skin deficient in fatty acid, ceramide and/or cholesterol comprising the steps of:
A) applying to skin in need of fatty acid, ceramide and/or cholesterol a composition comprising:
1) a hydrocarbon source comprising C₆ to C₁₈ fatty acid, ester and/or acylglycerol thereof;
2) at least one activator of a peroxisome proliferator-activated receptor;
3) an elongation source comprising C₂ to C₁₈ alcohol, saccharide, C₃ to C₈ polyol or a mixture thereof; and
4) from 0 to 5% by weight of niacin, niacinamide, inositol, hexanicotinate, picolinic acid, picolinamide, 1-methylnicotinamide chloride, N-acetyl-DL-methionine or a mixture thereof,
B) generating a C₂₀ to C₃₆ elongated fatty acid, lipid or both after applying the composition to the skin; and
C) restoring C₂₀ to C₃₆ fatty acid, ceramide and/or cholesterol levels in the skin to yield an equimolar mixture of the same wherein the C₂₀ to C₃₆ elongated fatty acid, lipid or both is/are produced in the stratum corneum of the skin.

4. The method for the personalized treatment of skin deficient in fatty acid, ceramide and/or cholesterol according to claim 3 wherein the hydrocarbon source is a C₆ to C₁₈ fatty acid.

5. The method for the personalized treatment of skin deficient in fatty acid, ceramide and/or cholesterol according to claim 4 wherein the fatty acid is palmitic acid.

6. The method for the personalized treatment of skin deficient in fatty acid, ceramide and/or cholesterol according to claim 3 wherein the elongation source is glycerol.

7. The method for the personalized treatment of skin deficient in fatty acid, ceramide and/or cholesterol according to claim 3 wherein the activator of a peroxisome proliferator-activated receptor is 12-hydroxystearic acid.

8. The method for the personalized treatment of skin deficient in fatty acid, ceramide and/or cholesterol according to claim 3 wherein the composition does comprise 1-methylnicotinamide chloride, N-acetyl-DL-methionine or both.

9. The method for the personalized treatment of skin deficient in fatty acid, ceramide and/or cholesterol according to claim 3 wherein the composition does comprise niacin, niacinamide, inositol, hexanicotinate, picolinic acid, picolinamide, or a mixture thereof.

10. The method for the personalized treatment of skin deficient in fatty acid, ceramide and/or cholesterol according to claim 3 wherein the composition further comprises a resorcinol, vitamin, sunscreen, antimicrobial agent, honokiol, mosquito repellant or a mixture thereof.

11. The method for the personalized treatment of skin deficient in fatty acid, ceramide and/or cholesterol according to claim 3 wherein the composition further comprises a quaternary ammonium salt.

12. The method for the personalized treatment of skin deficient in fatty acid, ceramide and/or cholesterol according to claim 3 wherein the composition is a leave-on, wash off or deodorant composition.

13. (canceled)

14. (canceled)

15. The method according to claim 3 wherein the composition further comprises eucalyptus oil, lavender oil, octopirox, zinc pyrithione, thymol, terpineol, cetrimonium chloride, benzethonium chloride, benzalkonium chloride, chloroxylenol, triclosan, cetylpyridinium, silver oxide, a nitrate, a sulfate, a phosphate, a carbonate, an acetate, a benzoate, honokiol, an isethionate, a taurate, a betaine, a sulfate based surfactant, a deodorant active or a mixture thereof.

16. The method according to claim 3 wherein the composition further comprises sapienic acid, palmitoleic acid, sphingosine, dihydrosphingosine, phytosphingosine, and 6-hydroxysphingosine, myeloid antimicrobial protein, cannabigerol and/or cannabidiol, retinol palmitate, retinol propionate, coumarin, farnesol, geraniol, climbazole, vanillin or a mixture thereof.
